(19) United States
(12) Patent Application Publication
(10) Pub. No.: US 2024/0285264 A1
(43) Pub. Date: Aug. 29, 2024

(54) ORAL FLUID SAMPLER FOR ANIMALS

(71) Applicant: Brian Michael Coyle, Canyon, CA (US)

(72) Inventors: Brian Michael Coyle, Canyon, CA (US); Alys Larsen, Canyon, CA (US)

(21) Appl. No.: 18/588,804

(22) Filed: Feb. 27, 2024

Related U.S. Application Data
(60) Provisional application No. 63/448,876, filed on Feb. 28, 2023.

(51) Int. Cl. A61B 10/00; G01N 33/52

(52) U.S. Cl. CPC A61B 10/0051; G01N 33/521; A01K 15/026; A61B 2503/40

(57) ABSTRACT

An oral fluid sample collection device (“sampler”) for animals includes a member with an external layer that can be held in an animal's mouth, a recessed layer that encloses an absorbent test strip, and an aperture in the external layer, wherein oral fluids pass through the aperture to be in fluid contact with the test strip, while the recessed layer is not in direct physical contact with the animal's mouth. A sampler including a compressible external layer. A sampler including a carrying case with test strip enclosures attached, the carrying case inserted in the member. A sampler including an internal layer that supports test strips which everts when an animal's mouth applies pressure to the external layer.

### CROSS-REFERENCE TO RELATED APPLICATIONS

The present application claims benefit under 35 USC § 119(e) of U.S. Provisional Patent Application Ser. No. 63/448,876 filed on 28 Feb. 2023. The present application is based on and claims priority from this application, the disclosure of which is hereby expressly incorporated herein by reference.

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

Existing sampling devices and methods for detection of periodontal disease (PD) pathogens tend to replace microscopy interpretation with digitized tools, but the basic sampling method remains absorbent paper placed into the ginvival pocket. To properly sample animals, they must be anesthetized, which imposes serious constraints on sampling. The sampled fluid contains microbes, microbial by-products, and inflammatory molecules, which can provide evidence of the degree and progression of the PD.

PD in dogs, cats, and other animals, including humans, attacks the junctional epithelium that plays a critical role in sealing off periodontal tissues from the oral environment. When a periodontal probe measures the depth of the gingival suculus, it stops when it reaches the junctional epithelium. PD sets in when the junctional epithelium starts to fail. This triggers inflammation, which leads to vascular leakage of the interstitial fluid between cells, called gingival crevicular fluid (GCF), found in the suculus. Polymorphonuclear leukocytes (PML) move to the injury site, passing from the subepithelial connective tissue through the junctional epithelium and into the gingival sulcus. If tissue loss continues, T-cells rapidly infiltrate. As the junctional epithelium is lost, a dense inflammatory fluid composed of plasma cells and macrophages (white blood cells that surround and kill microorganisms, remove dead cells, and trigger immune reactions) infiltrates the area. Periodontal disease destruction occurs from the host's uncontrolled immune responses, which causes chronic and exacerbated inflammation. The destruction of tooth-supporting alveolar bone involves molecules like interleukin-1 (IL-1), tumor necrosis factor alpha (TNF-a), matrix metalloproteinases (MMP) and prostaglandin E2 (PGE2).

Therefore, a succession of molecules carried in the GCF appear during PD progression, PML >T-cells >macrophages >IL-1, TNF-a, MMP, and PGE2. The infectious pathogens that cause PD, such as Porphyromonas gingivalis and Treponema denticola, produce neutral proteinases that also infiltrates the GCF. Studies find these proteinases levels are higher once sites become more severely inflamed sites. Finally, clinically significant PD is indicated by the presence of large numbers of periodontal pathogen bacteria themselves in the GCF, such as P. gingivalis, Tannerella forsythia and T. denticola (Ko et al. 2021, 1.)

DNA can be extracted from GCF and polymerase chain reaction (PCR) carried out, with primers for periodontal pathogenic bacteria. Cytokines like IL-1 and IL-6 are determined by standard ELISA kits. The GCF is sampled with paper strips or paper points placed for at least 30 seconds into a gingival pocket. With most non-sedated animals, this cannot be done, or done well, by non-specialists. Many veterinary staff are appropriately concerned that they will be bitten by animals during oral exams (Oxley et al. 2024, 2.) Saliva contains GCF, diluted. But the farther removed from an infection site, the more diluted the GCF becomes. For humans, WO Patent App. 2018/005335 A1 of The Proctor & Gamble Co. (P1) describes a brush to obtain GFC while swabbing back and forth at the gum line. Since gums are sensitive, this would antagonize an animal. For animals, the solution is to obtain oral fluid close to the sites of potential infection, using a device that an animal can hold in its teeth, can bite, or can chew.

### SUMMARY OF THE INVENTION

The present invention provides an apparatus for detecting PD molecular patterns, cellular receptors, proinflammatory mediators, and target cells and tissues, present in the fluid near the gums and teeth in an animal's mouth.

The inventor of the present application has filed previous U.S. Provisional Patent Application Ser. No. 63/448,876 filed on 28 Feb. 2023. The above-referenced patent application is hereby incorporated by reference into the present application.

In one aspect of the present disclosure, an oral fluid sampler device (“sampler”) that an animal bites, chews, or otherwise holds in its mouth has recessed surfaces to hold test strips, to enable safe, error free sampling of oral fluids. According to the various embodiments, the sampler is formed by having a enclosure that holds a test strip, which is in a recessed position relative to the outermost surface of the sampler, and the animal's oral fluids flow through an aperture in the outermost surface to flowingly contact the test strip, and the animal's mouth is only in direct contact with the outermost surface, leading to the advantage that the test strip is adequately protected during use.

In an embodiment a carrying structure is slidingly mounted in the sampler, with one or more test strips rigidly attached therein, and after use the carrying structure is removed and the test strips placed in a collection receptacle for additional processing.

In an embodiment the test strip is attached to the enclosure, with an additional material at or near the outermost surface over the test strip, the additional material forming a cap that selectively limits oral fluid that contacts the test strip.

In an embodiment the test strip is attached to the enclosure, with a compound or compounds configured on or within a material or component at or near the outermost surface, the material or component releasing the compound or compounds that selectively increase migration of certain oral molecules into contact with the test strip.

In an embodiment the sampler is composed of materials with various hardness ratings.

An embodiment is composed of materials with more than one Shore hardness rating. The outermost surface opposite to an animal's oral environment has a Shore A hardness between 0 and 50, and an inner section of the device has a Shore A hardness between 30 and 70. An embodiment has an outermost surface with a Shore 00 rating of 10 to 70.

An embodiment is composed of materials with more than two hardness ratings. An embodiment has multiple hardness levels, from most pliable to most firm, with an outermost surface opposite to animal's oral environment with a hardness that is most pliable, a section that holds the test strip with a hardness that is most firm, and an inner section has a hardness that is moderate, in between the most pliable and most firm.

An embodiment is composed of materials with progressively higher hardness ratings, with the lowest hardness rating on the device section closest to the outermost surface, with increasing hardness ratings in materials that are closer to the sampler center.

An embodiment is configured with an empty area in the center of the sampler.

An embodiment is configured with an outermost surface opposite to an animal's oral environment that contains within it various hardness ratings, to induce an animal to masticate the device. An embodiment with various hardness ratings in the outermost surface of the device is also composed of materials further inside the device that have a homogeneous hardness rating.

An embodiment is configured with ridges on its outer surface. In a further embodiment, the sampler is configured with elastic ridges that respond to applied pressure by compression and bending outward from the axis of force, reducing the ridge height and bringing the test strip attached to the enclosure closer to the outermost surface.

An embodiment is configured to perform an eversion when an animal applies pressure on the device, with the surface containing the enclosure cavitating when pressure is applied to the device, the eversion bringing the test strip closer to the outermost surface. The expansion of the surface containing the enclosure pushes the sample paper outward.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which embodiments of the invention are illustrated.

### BRIEF DESCRIPTION OF THE DRAWINGS

The above objects and purposes and the description of the prevent invention as set forth hereinafter will be better understood with reference to the accompanying drawings, in which:

FIG. 1 is a front view of the sampler.

FIG. 2 illustrates the sampler as held in the mouth of an animal.

FIG. 3 illustrates the sampler with raised ridges on the first surface.

FIG. 4 is a schematic illustration of a set of ridges with sidewalls that bow under compression.

FIG. 5 illustrates a carrying structure containing test strips being removed from the sampler.

FIG. 6 illustrates an enclosure including a test strip being removed from the carrying structure.

FIG. 7 illustrates a sampler with multiple layers each having a different hardness rating.

FIG. 8 illustrates a sampler with an outermost layer containing areas with different hardness ratings.

FIG. 9 illustrates a sampler with external surface visible within which an internal surface contains the test strips.

FIG. 10 illustrates the eversion of the internal surface exposing the test strips when pressure is applied to the sampler.

### DETAILED DESCRIPTION

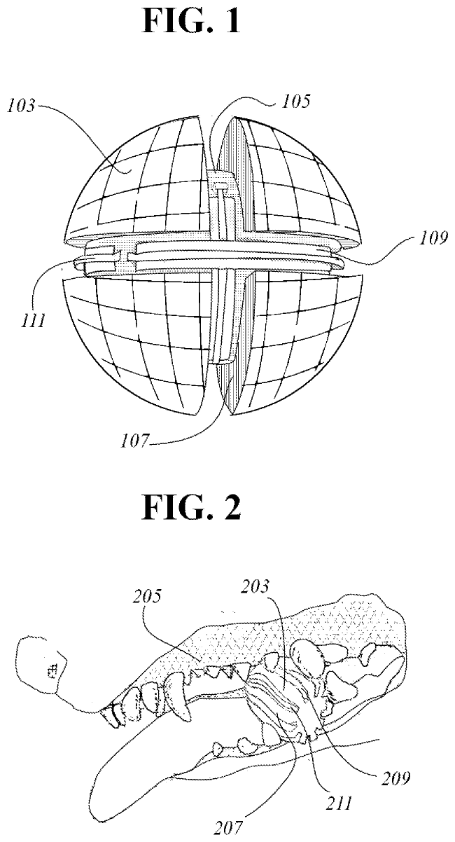

FIG. 1 illustrates an oral fluid sampling device (“sampler”). Outermost surface 103 is formed of at least one layer of a flexible polymer, which may be held in the mouth of an animal. Internal layer 105 forms the basis of an enclosure 107 which holds a test strip 109. A compatible mechanism in the form of a bracket or elastic band 111 is placed on or over the test strip 109 to keep it in position.

FIG. 2 illustrates the sampler 203 in the mouth 205 of an animal. Elevated flexible ridges 207 on the device 203 protect the enclosures 209 that contain test strips 211. The flexible ridges 207 are compressed by the animal's mouth 205.

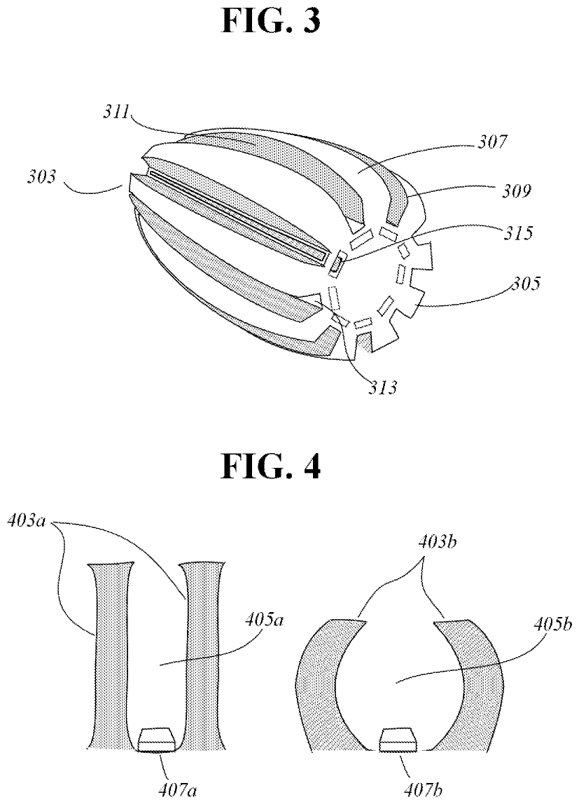

FIG. 3 illustrates the sampler 303 with flexible ridges 305. The outermost surface 307 is directly engagable by the mouth of the animal. Enclosures 309 are formed with sidewalls 311 whose size may be varied to accommodate various lengths and diameters of the device. The interior surface 313 of each enclosure partially encloses a space that contains the test strip 315.

FIG. 4 illustrates an enlarged schematic view of a pair of uncompressed sidewalls 403a and a pair of compressed sidewalls 403b, each pair of sidewalls 403a and 403b being vertically spaced around an enclosure 405a and 405b with a test strip 407a and 407b. The sidewalls 403a show the condition when the sampler is not under pressure in an animal's mouth. When pressure is applied to the sampler proximal to the vertical axis, sidewalls 403b are deformed outwardly.

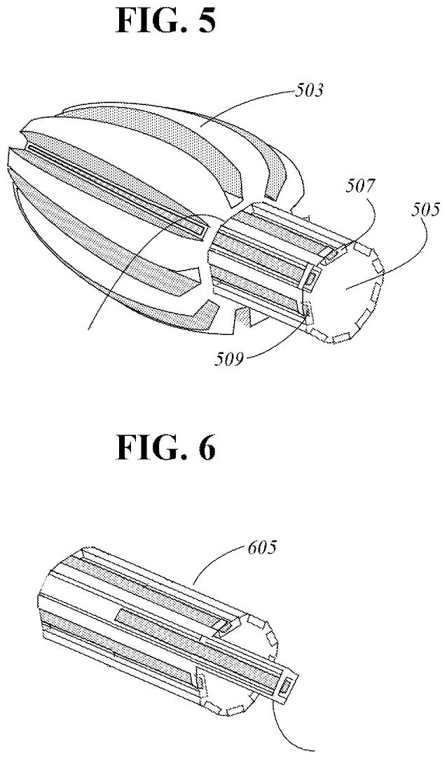

FIG. 5 illustrates the sampler 503 with a carrying case 505 that holds enclosures 507 that each contain a test strip 509. The body of the carrying case 505 is configured to grip the device internal body 511 when inserted fully into it. A person can apply a force that slideably removes the carrying case, as illustrated.

FIG. 6 illustrates the removed carrying case 605, with an enclosure 607 released from its position and withdrawn from the carrying case 605.

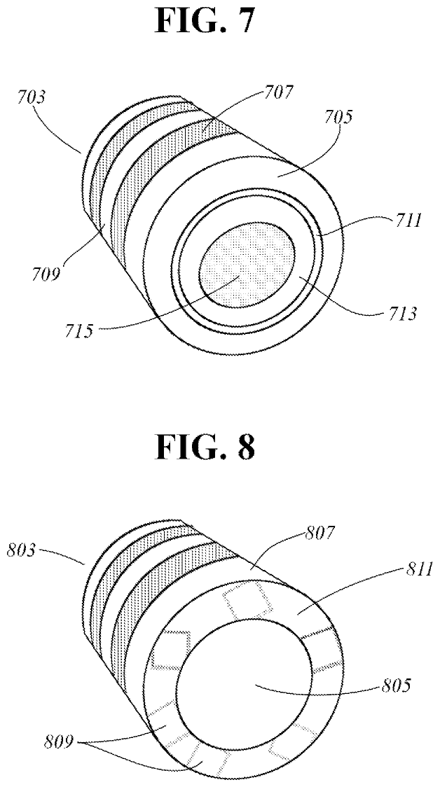

FIG. 7 illustrates the sampler 703 with an outermost layer 705 incorporating enclosures 707; the outermost layer 705 has an outer surface 709; the outermost layer 705 has a hardness rating less than that of a second internal layer 711 that supports a surface with test strips (not visible.) A first internal layer 713 has a different hardness rating than the second internal layer 711. The core of the device 715 may be hollow. The second internal layer 711 provides support for test strips to prevent applied pressure from deforming the test strips.

Although only three layers and a core are shown in FIG. 7, it should be appreciated that the sampler may have more than three layers of hardness, in an embodiment going from softest to hardest going inwardly from the outer surface. In another embodiment the innermost layer, or material that may form the core, may be composed of a softer material than the outer layer, which can be beneficial for controlling deformation forces applied by the animal's mouth.

FIG. 8 illustrates another embodiment of the sampler 803 that includes an inner layer 805 that has a different hardness rating than at least some material in the outermost layer 807. The outermost layer 807 includes units of material 809 that have a higher hardness rating than the material 811 of outermost layer that they are embedded in.

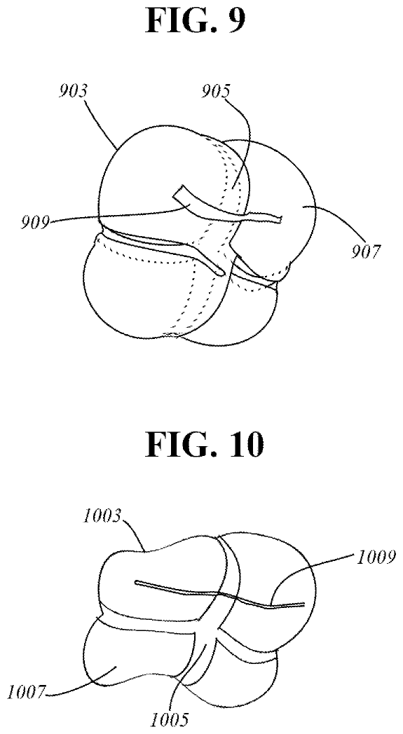

FIG. 9 illustrates the sampler 903 in which enclosures containing test strips are disposed on interior surfaces 905 when in a closed configuration, the interior surfaces 905 having been folded into the device when prepared, so that primarily the outermost surface 907 is exposed; this closed configuration is maintained by an elastic attachment 909.

FIG. 10 illustrates the sampler 1003 when external pressure is applied; the elastic attachment 1009 and the outermost surface 1007 extend elastically, thereby allowing the interior surfaces 1005 to evert and be exposed to oral fluid flow.

It can readily be seen that the oral fluid sampling process in accordance with the invention readily allows one to attain the advantage of safely exposing test strips to oral fluid flow in the mouth of an animal, by use of device that isolates test strips in enclosures from direct physical contact with the mouth of the animal, while oral fluid can be introduced to the test strips through apertures in the device.

It will thus be seen that the objects set forth and made apparent from the description above are efficiently attained and, since variations may be made carrying out the invention set forth without departing from the spirit and scope of the invention, all descriptions and drawings shall be interpreted as illustrative and not in a limiting sense.

The following claims are intended to cover all of the generic and specific features of the invention herein described and all statements of the scope of the invention which, as a matter of language, might be said to fall there between.

1. A sampler apparatus for collecting oral fluids in an animal, comprising:
a first surface in direct contact with a mouth of the animal;
a second surface configured with an enclosure to hold at least one removably attached test strip that absorbs oral fluids;
an aperture in the first surface permits oral fluids to flow from the mouth of the animal to make flowing contact with the at least one replaceable test strip;
wherein the aperture separates the at least one replaceable test strip on the second surface from direct contact with the mouth of the animal.

2. The sampler apparatus of claim 1, wherein the enclosure has a mechanism to hold the test strip in place.

3. The sampler apparatus of claim 1, wherein the second surface is a carrying structure configured to hold the test strip; the carrying structure is configured to be removed from the sampler apparatus after use.

4. The sampler apparatus of claim 1, wherein a cap is configured to cover the test strip, wherein the cap comprises a material that selectively limits the oral fluids that flowingly contact the test strip.

5. The sampler apparatus of claim 1, wherein a material proximal to the first surface releases at least one compound to increase the absorption of the oral fluids in the test strip.

6. The sampler apparatus of claim 1, wherein the sampler apparatus includes multiple layers circumferentially distributed around a core; comprising at least a first layer including a first material having a first hardness rating, and at least a second layer including a second material having a second hardness rating, wherein the first hardness rating and the second hardness rating are different.

7. The sampler apparatus of claim 6, wherein the at least first layer includes the first surface and the at least second layer includes the second surface, and the second rating is higher than the first hardness rating.

8. The sampler apparatus of claim 6, wherein the core is hollow.

9. The sampler apparatus of claim 1, wherein the first surface is composed of ridges extending in a raised position.

10. The sampler apparatus of claim 9, wherein the ridges compress when pressure is applied by the mouth of the animal, bringing the second surface closer to the first surface.

11. A method for sampling oral fluids in the mouth of an
animal; providing a member the animal holds in its mouth;
providing a test strip attached to a flexible internal surface of the member;
the animal applies a pressure to the member;
the pressure opening an external surface of the member; everting the flexible internal surface into the opening to expose the test strip to the oral fluids.
